(19) United States
(12) Patent Application Publication
(10) Pub. No.: US 2005/0070830 A1
(43) Pub. Date: Mar. 31, 2005

(54) Apparatus to serve as a reminder for posture improvement

(76) Inventor: Robert Jordan Schultz, Rye, NY (US)

Correspondence Address: REED SMITH, LLP;ATTN: PATENT RECORDS DEPARTMENT, 599 LEXINGTON AVENUE, 29TH FLOOR, NEW YORK, NY, 10022-7650, US

(21) Appl. No.: 10/949,752

(22) Filed: Sep. 24, 2004

Related U.S. Application Data
(60) Provisional application No. 60/506,291, filed on Sep. 27, 2003.

(51) Int. Cl. A61F 5/00; A61L 15/00

(52) U.S. Cl. 602/19

(57) ABSTRACT

Disclosed is an apparatus that serves to alert an individual to a change in the individual's posture. A responder that provides an alerting signal to the wearer of the apparatus is mounted on an elastic band that is worn by the individual. When the individual's posture is correct, there is no alerting signal. When the individual's posture changes, and in particular by slouching, the elastic band stretches, a switch is then closed in the responder thereby completing an electrical circuit which generates an alerting signal such as a small buzz or tone to the wearer. In response to this signal, the wearer can correct posture and thus turn off the alerting signal.

### CROSS REFERENCE TO RELATED APPLICATIONS

This application claims priority under 35 U.S.C. §119(a) to U.S. Provisional Application Ser. No. 60/506,291, filed on Sep. 27, 2003, the disclosure of which is hereby incorporated by reference.

### FIELD OF THE INVENTION

The present invention relates generally to an apparatus that is worn by an individual to be used as a reminder that an individual is slouching. Over time the maintaining of good posture will become natural.

### BACKGROUND OF THE INVENTION

People often find themselves slouching when standing, walking and sitting, especially now in the days of the computer. When someone is conscious of his or her poor position they bring their bodies into a more erect attitude. However, shortly thereafter when they are otherwise preoccupied they once again assume the stooped position.

There are many varieties of braces for holding the spine in proper position. However, these braces are not intended for, and do not lead to muscle strengthening, and thus ultimately do not lead to the ability for one to actively hold proper posture. These braces are generally designed to hold a physically challenged individual in a proper attitude and prevent further worsening of the person's spinal curve, or to allow a paralyzed physically challenged individual to function in an upright position. Examples of these braces are devices used for persons with curvatures of the spine as scoliosis, to prevent further worsening of the curvature while they are growing, and those with paralytic muscles who require support to hold them upright. In fact, devices such as braces that hold an individual in a fixed or rigid position, can eventually lead to weakening of the support muscles. Thus it is far better for people with otherwise normal spines, but inclined to slouching, to strengthen ones own muscles to maintain proper posture.

Accordingly, it is a major purpose of this invention to provide a means to improve posture or position by strengthening the muscles of the user and/or positively affecting the bony architecture.

The present device is not a brace, nor an apparatus that holds an individual in a fixed position, but a device that is worn and used as a reminder, so that if an individual is slouching a signal is produced, and the person is reminded of the poor posture so they can voluntarily assume the proper posture. Over time the maintaining of good posture will become natural.

### BRIEF DESCRIPTION

The apparatus and method of the present invention can provide an efficient way of improving a person's posture. In particular, the invention is directed to an apparatus that is worn to alert an individual when they are slouching, so that they can voluntarily straighten up and eventually improve their posture.

More specifically, one embodiment of this invention provides an apparatus for reminding a person of a poor posture position. It includes a stretchable, flexible, or otherwise malleable elastic component that when bent or stretched or otherwise deformed in response to poor posture will provide a signal to the user. The materials for this component can be of materials now known or later developed.

The signal is provided by a known type of electric circuit having a switch, a power source and an audible or vibrating signal device, such as a speaker, which is turned on by switch closure.

There are many materials that contain elastic properties. As used herein, the term “flexible elastic band or component” means any variety, brand or substance that can deform and have the ability to return to its original shape.

When the flexible elastic component is in, or returned to, its original position the signal is quiet. When the wearer slouches the flexible elastic component is deformed, a switch closes and a signal is produced until the user straightens up.

In this invention, the apparatus for reminding the wearer of slouching is comprised of a harness that secures the apparatus to the individual. The harness holds a flexible elastic band that deforms when the body is in malposition, and returns to its original form when the deforming force is removed. A responder mounted on the electric band responds to the deformation by providing a signal that the wearer can sense.

The harness that secures the apparatus to the wearer may be composed of two adjustable shoulder supports or straps connected by the flexible elastic band. The adjustable shoulder straps fit about the shoulders of the wearer who places his or her arms through the loops of the straps. The flexible elastic band is positioned on the upper back and set in its relaxed, shortened position for correct posture. The harness is adjustable, to account for variations in individual body structure. The flexible elastic band may be of various lengths to accommodate the different sizes of the user. If the wearer slouches, the flexible elastic band is stretched generating an impulse that will ultimately signal the wearer by means of an alerting device and may continue until the flexible elastic band again assumes its original or relaxed state.

### BRIEF DESCRIPTION OF THE FIGURES

FIG. 1 schematically shows a first embodiment of the device of this invention in which the responder which provides an alerting signal is mounted on an elastic band held on the back of an individual by a shoulder and torso harness.

FIG. 2 is an electrical schematic showing of the responder in a state when the wearer's posture is correct and no signal is provided.

FIG. 3 is a schematic illustration showing the responder in the state where the wearer's position is incorrect and the band on which the responder is mounted is stretched in response to the wearer's slouch so that a switch contact is made thereby causing the alerting signal to be generated.

### DESCRIPTION OF THE PREFERRED EMBODIMENTS

As shown in the FIGs, an apparatus 10 is provided that can be worn and used to alert an individual when they are slouching, so that they can straighten up and eventually improve their posture. Over time as the appropriate muscles are strengthened, the maintaining of good posture will become natural.

The apparatus shown is comprised of a harness 11 that secures the apparatus to anatomic parts of the individual. The harness 11 supports a flexible elastic band 12 that deforms when the body is in malposition (slouched), and that returns to its original state when the deforming force is removed, namely, when good posture is attained.

A responder 14 provides an alerting signal when the band 12 stretches in response to the wearer's slouching. The signal notifies the wearer that they are not in proper position. In the embodiment shown, the responder 14 is mounted on the elastic band 12.

The responder is essentially a switch 16 and a signaling device. The signaling device has a power supply 18, an electric signal generator 20 and a speaker 22. In the embodiment shown, the switch 16 is normally open. When the wearer slouches, the switch 16 closes, connecting the power source 18 to the signal generator 20. The generator 20 provides an output to the speaker 22 resulting in an audible sound. This type of responder design is well known and exists for, example, in audible greeting cards.

The responder 14 can provide a tone, a vibration or any other type of dispensable signal.

The switch 16 can be mechanical or piezo-electric or any other type of switch.

The speaker 22 can be replaced with any device that provides a signal the wearer can sense.

The signaling device comprised of power supply 18, signal generator 20 and speaker 22, can be mounted on the user at any location and need not be on the band 12.

The embodiment described has all the components of the responder 14 mounted on the elastic band 12. The components of the responder, other than the switch 16, can be mounted separate from the elastic band. In some circumstances, where the wearer requires two separate alerting bands to respond to two related postural positional corrections, it might be desirable to use two switches and a single set of power source, generator and sensor.

Depending on the situation, it might also be desirable to have two separate responders providing two different signals in order to alert an individual to which of two separate positional or posture alignments need to be corrected.

It should also be understood that certain piezo-electric devices could be used, instead of a mechanical switch, to provide an electric signal, in response to posture change, which electric signal can be used to energize a speaker or the like.

Accordingly, this invention is set forth in the claims. It should be understood that it is not limited to the particular embodiment shown or embodiments described.

One illustrative embodiment of a posture reminding apparatus is shown in the FIGs.

In operation, the posture reminding apparatus is donned by the wearer by placing the arms through the shoulder straps that are positioned about both shoulders, with the flexible elastic band 12 set across the upper back in the area of the shoulder blades. The wearer should voluntarily assume the proper posture and adjust the posture-reminding apparatus using an adjusting mechanism 28, such as a Velcro connection, so that the band 12 is in its relaxed state. Apart from the switch 16, the rest of the responder 14 can be placed anywhere on the body that is comfortable to the wearer. As shown, it can be installed somewhere within or on the band 12.

In other embodiments (not shown) the posture reminding apparatus is secured over the shoulders by a support or any other shoulder retaining mechanism and to the pelvis by pelvic bands or any other pelvic attachment thus supporting the posture-reminding apparatus above and below the spine.

In another embodiment (not shown), the flexible elastic band can run along the spine linking the upper and lower harness supports.

The flexible elastic band 12 can be placed anywhere on the body that can determine posture or position. For example, the band 12 can be attached to the front of the chest. In this embodiment, when the body is in proper position, the band 12 is stretched and no impulse is generated. If the person slouches, the band contacts to close the switch and generate the sensible signal.

While the foregoing description and drawings represent the presently preferred embodiments of the invention, it should be understood that those skilled in the art will be able to make changes and modifications to those embodiments without departing from the teachings of the invention and the scope of the claims.

For example, the flexible elastic component can also be configured to generate an impulse when the component is in its original or relaxed attitude. When the flexible elastic component is stretched or otherwise deformed, the impulse is in a quiet state.

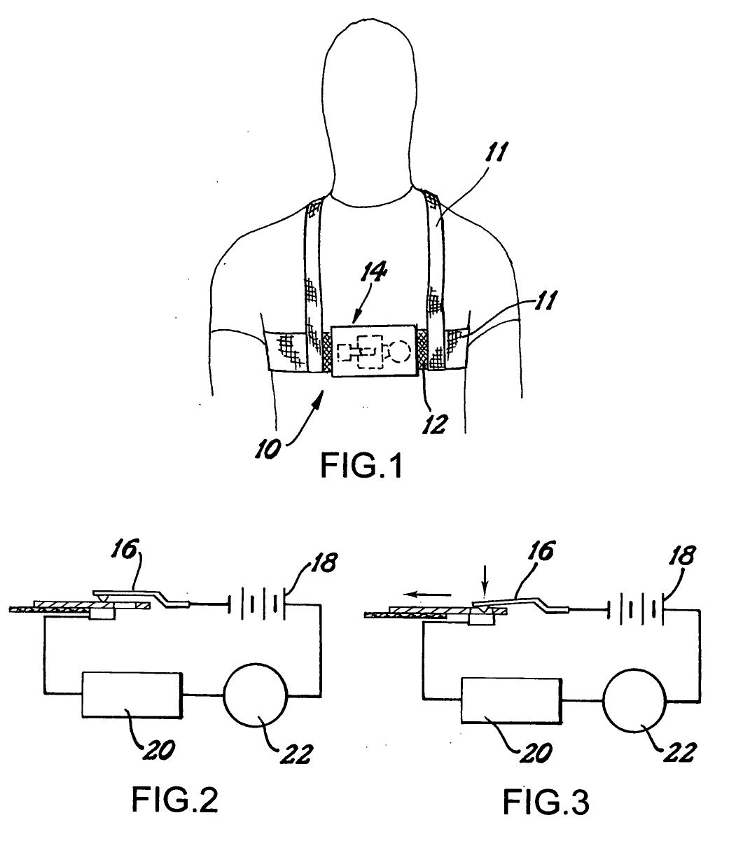

For example, in another embodiment of the present invention, two shoulder supports can secure the posture reminding apparatus to the upper body, and two pelvic stays or supports fix the lower part of the posture reminding apparatus to the pelvis below the spine. The flexible elastic component is in the shape of a central longitudinal connector running in line with the spine and spans and links the upper and lower supports. As in the FIG. 1 embodiment, when the person slouches the flexible elastic component will bend, stretch or otherwise be deformed setting off an impulse that will signal the wearer by means of the alerting device or any other method, and continue until the flexible elastic component assumes its original or neutral position.

In another embodiment, the flexible elastic component can be directly bonded to an individual's body and secured by an adhesive, other bonding agent or secured in any conventional manner now known or later developed.

The flexible elastic component can be attached to any part of an individual's body that is capable of detecting poor posture or other body attitude by causing the flexible elastic component to change state and then produce a signal which the wearer can sense.

1. Apparatus for alerting an individual to a predetermined change in posture comprising:
an elastic member adapted to be worn on the body, said member having two states, a first normal state when the individual's posture has a first state and a second signal state when the individual's posture has a second state, and
a responder coupled to said elastic member to provide an alerting signal to the wearer of the elastic member in response to said second state of said elastic member.

2. The apparatus of claim 1 wherein said responder comprises:
a switch coupled to said elastic member, said switch having a first state in response to said first state of said elastic member and a second state in response to said second state of said elastic-member, and
a signaling device responsive to the state of such switch to provide said alerting signal.

3. The apparatus of claim 2 wherein: said signaling device is carried by said elastic member.

4. The method of alerting an individual to a predetermined change in posture comprising the steps of:
positioning an elastic member on the body, said member having two states, a first normal state when the individual's posture has a first state and a second signaling state when the individual's posture has a second state, and
coupling a responder to said elastic member to provide an alerting signal to the wearer of the elastic member in response to said second state of said elastic member.

5. The method of claim 4 wherein: said step of coupling comprises the steps of:
mounting a switch on said elastic member, said switch having a first state in response to said first state of said elastic member and a second state in response to said second state of said elastic member, and
providing a signaling device responsive to the state of such switch to provide said alerting signal.

6. The method of claim 4 wherein: said step of coupling comprises the step of:
mounting said responder on said elastic member.
